(19) United States
(12) Patent Application Publication
(10) Pub. No.: US 2003/0143312 A1
(43) Pub. Date: Jul. 31, 2003

(54) Baking adjuvant

(75) Inventors: Dov Tamarkin, Maccabim (IL); Meir Eini, Ness Ziona (IL); Micha Peled, Rehovet (IL)

Correspondence Address: HALE AND DORR, LLP, 60 STATE STREET, BOSTON, MA, 02109

(73) Assignee: Thixo Ltd.

(21) Appl. No.: 10/174,163

(22) Filed: Jun. 18, 2002

Related U.S. Application Data
(63) Continuation-in-part of application No. PCT/IL01/00024, filed on Jan. 10, 2001.
(63) Continuation-in-part of application No. 09/526,509, filed on Mar. 16, 2000, now Pat. No. 6,348,229.
(63) Continuation-in-part of application No. 09/883,721, filed on Jun. 18, 2001.
(63) Continuation-in-part of application No. PCT/IL01/00024, filed on Jan. 10, 2001.
(60) Provisional application No. 60/299,141, filed on Jun. 18, 2001.

(51) Int. Cl. A23D 7/00

(52) U.S. Cl. 426/601

(57) ABSTRACT

An oil composition including a mixture of an oil and a baking adjuvant is provided, which can be used in the same way as conventional fats in baking to provide baked goods having improved volume, texture, and dietary value. Useful baking adjuvants include fatty acids having a molecular weight of at least 200 Da, fatty alcohols having a molecular weight of at least 200 Da, and mixtures thereof.

### CROSS-REFERENCE TO RELATED APPLICATIONS

This application is a continuation-in-part of PCT/IL01/00024, filed Jan. 10, 2001, which is a continuation-in-part of application Ser. No. 09/526,509, filed Mar. 16, 2000, now U.S. Pat. No. 6,348,229. This application is also a continuation-in-part of application Ser. No. 09/883,721, filed Jun. 18, 2001, which is a continuation-in-part of PCT/IL01/00024, filed Jan. 10, 2001. This application also claims priority to provisional application Ser. No. 60/299,141, filed Jun. 18, 2001.

### BACKGROUND

1. Field of the Invention

The invention relates to oil compositions including a baking adjuvant for improving the quality of baked goods. In particular, the invention relates to cakes, muffins, breads, cookies, and the like, having improved volume and texture and an improved health profile.

2. Discussion of Related Art

Fats are prominent constituents of baked goods. Liquid, unsaturated oils occasionally are used in baking. However, the use of semi-solid and solid fats, generally saturated fats, in baking is most common, especially in industrial preparation. Semi-solid and solid fats can be derived from natural sources. Such fats can be isolated from animal sources (e.g., lard and milk fats) or from plants, such as palm and coconut. It is well known, and was further stated by the Food and Drug Administration (FDA), that the intake of saturated fat is linked to high blood cholesterol, which in turn is linked to increased risk of coronary heart disease (CHD) (See “Lowering Cholesterol,” FDA Consumer, March, 1994; “A Consumer's Guide to Fats,” FDA Consumer, May, 1994). It has been established that the cardiovascular risk of saturated fats is inversely correlated with the length of their fatty acids: lauric acid (C12)>palmitic acid (C14)>arachydic acid (C16)>stearic acid (C18). Plant-derived fats are rich in lauric acid and palmitic acids.

Another way to obtain semi-solid and solid fats is partial or full hydrogenation of oils, which become semi-solids due to the hydrogenation process. However, because of this chemical transformation, the oils become saturated and lose their healthy properties. Partial hydrogenation also results in the formation of “trans” fatty acids, which have recently been shown to possess a plurality of adverse properties. Structurally, trans fatty acids are similar to saturated fatty acids, and hence they influence cell membranes in the same way. Saturated fatty acids elevate LDL cholesterol levels by inhibiting the removal of cholesterol from the blood by inhibiting LDL receptors. Trans fatty acids also can raise LDL cholesterol levels in the blood. The Food and Drug Administration recently has stated that consumption of saturated oils containing trans fatty acids contributes to increased blood LDL cholesterol (“bad” cholesterol) levels, which increase the risk of coronary heart disease (U.S. Department of Health and Human Services, Press Release, Nov. 12, 1999). At high levels of intake, trans fatty acids can also reduce blood levels of HDL cholesterol (the “good” cholesterol) and interfere with the metabolism of essential fatty acids. Trans fatty acids apparently also elevate lipoprotein(a), a risk factor for heart disease. In the Nurses Health Study in Boston, it was reported that intake of trans fatty acids was associated with an increased risk of coronary heart disease. Another example relates to trans linoleates, which are devoid of essential fatty acid activity. Animal studies have shown that they retard growth more than diets deficient in essential fatty acids. Trans, trans 18:2-w6 linoleic acid decreases the conversion of linoleic to gamma-linolenic acid in liver microsomes (Handbook of Lipids in Human Nutrition, Spiller, ed. (1996), p. 92). Trans fatty acids can enter fetus and breast milk. They can be incorporated into a child's developing brain instead of more beneficial fatty acids. A recent European epidemiological study also has revealed an association between ingestion of sizable amounts of trans fatty acids and the prevalence of asthma, allergic rhino conjunctivitis, and atopic dermatitis (Lancet 353:2040-41 (1999)).

Other oily substances that can substitute for oils, semi-solid, and solid fats are the “fat replacers.” Fat replacers are classified by their chemical nature. They include monoacylglycerols, diacylglycerols, and special triacylglycerols having specific combinations of short-, medium-, and long-chain fatty acids. Another class of fat replacers includes sucrose esters and related compounds.

Improving the fat-related health profile of baked goods, as well as improving the texture and increasing the volume of such products is generally desirable. Therefore, a need exists for new oil compositions for producing improved baked goods.

### SUMMARY

Oil compositions including baking adjuvants are provided for use in batters and doughs for producing baked goods. The oil compositions are useful for producing baked goods having improved volume and texture compared to similar baked goods made using the same recipe but with conventional oil or shortenings. The oil compositions also are useful for producing baked goods having reduced caloric value and baked goods having a cholesterol-lowering effect.

In one aspect, the invention provides an oil composition for use in baking comprising a mixture of between about 70% and about 99.95% of an oil and between about 0.05% and about 30% of a baking adjuvant selected from the group consisting of fatty alcohols having a molecular weight of at least 200 Da, fatty acids having a molecular weight of at least 200 Da, and combinations thereof. In certain embodiments, the oil is selected from the group consisting of liquid oils, semi-solid and solid fats, fat replacers, and combinations thereof in any proportion. In particular embodiments, the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil. In specific embodiments, the oil comprises an oil or fat selected from the group consisting of a partially or fully hydrogenated oil, an animal-derived fat, margarine, coconut oil, palm oil, palm kernel oil, butter fat, and cocoa fat. In certain embodiments, the oil comprises a fat replacer selected from the group consisting of diglycerides, triglycerides, combinations of short- and long-chain fatty acids, sucrose esters, and edible indigestible hydrophobic materials. In particular embodiments, the oil in the oil composition comprises at least about 25% of a fat replacer having a caloric value of less than 6.9 kcal/g. Another aspect of the invention provides a baked good comprising such an oil composition. In specific embodiments, the baked good is selected from the group consisting of cake, muffin, tart, bread, bun, bagel, cookie, biscuit, wafer, pie, pizza, pita, doughnut, pancake, cracker, and pastry.

In certain embodiments, the adjuvant in the oil composition comprises a fatty alcohol having a molecular weight of at least about 200 Da. In particular embodiments, the fatty alcohol has between about 15 and about 50 carbon atoms in its hydrocarbon chain. In specific embodiments, the fatty alcohol is selected from the group consisting of cetyl alcohol, stearyl alcohol, arachidyl alcohol, behenyl alcohol (docosanol), octacosanol, and 1-triacontanol. In certain embodiments, the fatty alcohol has a branched hydrocarbon chain having at least one alkyl group side chain. In particular embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In specific embodiments, the fatty alcohol has at least one additional hydroxyl group linked to the hydrocarbon chain. In certain embodiments, the fatty alcohol has at least one double bond in the hydrocarbon chain.

In some embodiments, the adjuvant comprises a fatty acid having a molecular weight of at least about 200 Da. In certain embodiments, the fatty acid has between about 15 and about 50 carbon atoms in its hydrocarbon chain. In particular embodiments, the fatty acid is selected from the group consisting of hexadecanoic (palmitic) acid (C16), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30). In specific embodiments, the fatty acid has a branched hydrocarbon chain having at least one alkyl group side chain. In certain embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In some embodiments, the fatty acid has at least one additional hydroxyl group linked to its hydrocarbon chain. In particular embodiments, the fatty acid has at least one double bond in its hydrocarbon chain.

In some embodiments, the adjuvant in the oil composition comprises at least one fatty alcohol and at least one fatty acid in a ratio between about 2:8 and about 8:2. Another aspect of the invention provides a baked good comprising such an oil composition. In specific embodiments, the baked good is selected from the group consisting of cake, muffin, tart, bread, bun, bagel, cookie, biscuit, wafer, pie, pizza, pita, doughnut, pancake, cracker, and pastry. In certain embodiments, the fatty alcohol is stearyl alcohol and the fatty acid is behenic acid. A further aspect of the invention provides a baked good comprising such an oil composition. In specific embodiments, the baked good is selected from the group consisting of cake, muffin, tart, bread, bun, bagel, cookie, biscuit, wafer, pie, pizza, pita, doughnut, pancake, cracker, and pastry.

In some embodiments, the oil composition comprises between about 80% and about 99.9% of the oil, and between about 0.05% and about 20% of the adjuvant. In particular embodiments, the oil composition comprises between about 0.05% and about 10% of the adjuvant. In certain embodiments, the oil composition further comprises at least one additive selected from the group consisting of emulsifiers, stabilizers, anti-oxidants, flavors, and colorants.

In another aspect, the invention provides a batter or dough comprising the oil composition. In yet another aspect, the invention provides a baked good comprising the oil composition. In some embodiments, the baked good is selected from the group consisting of cake, muffin, tart, bread, bun, bagel, cookie, biscuit, wafer, pie, pizza, pita, doughnut, pancake, cracker, and pastry. In certain embodiments, the baked good has a reduced caloric value. In particular embodiments, the baked good has a cholesterol-lowering effect. In still another aspect, the invention provides an icing comprising the oil composition. In a further aspect, the invention provides a baking kit comprising the oil composition and additional baking ingredients. In certain embodiments, the additional baking ingredients comprise ingredients for making at least one baked good selected from the group consisting of cake, muffin, tart, bread, bun, bagel, cookie, biscuit, wafer, pie, pizza, pita, doughnut, pancake, cracker, and pastry.

Another aspect of the invention provides an adjuvant-oil concentrate for use in baking comprising between about 5% and about 50% of an oil and between about 50% and about 95% of a baking adjuvant selected from the group consisting of fatty alcohols having a molecular weight of at least 200 Da, fatty acids having a molecular weight of at least 200 Da, and combinations thereof. In some embodiments, the adjuvant comprises at least one fatty alcohol and at least one fatty acid in a ratio between about 2:8 and about 8:2. In certain embodiments, the fatty alcohol is stearyl alcohol and the fatty acid is behenic acid. Another aspect of the invention provides a baking kit comprising the concentrate and additional baking ingredients. In certain embodiments, the additional baking ingredients comprise ingredients for making at least one baked good selected from the group consisting of cake, muffin, tart, bread, bun, bagel, cookie, biscuit, wafer, pie, pizza, pita, doughnut, pancake, cracker, and pastry.

In another aspect, the invention provides a method of reducing or preventing an increase in a blood cholesterol level comprising administering to a subject in need thereof an effective amount of the oil composition. In still another aspect, the invention provides a method of treating or preventing hyperlipidemia or hypercholesterolemia comprising administering to a subject in need thereof an effective amount of the oil composition. In yet another aspect, the invention provides a method of promoting weight loss or preventing weight gain comprising administering to a subject in need thereof an effective amount of the oil composition.

A further aspect of the invention provides an emulsion comprising between about 40% and about 90% of the oil composition and between about 10% and about 60% water. In certain embodiments, the emulsion further comprises at least one additive selected from the group consisting of emulsifiers, stabilizers, flavors, and colorants.

Another aspect of the invention provides a batter or dough comprising an oil composition, said oil composition comprising a mixture of an oil and an adjuvant selected from the group consisting of fatty alcohols having a molecular weight of at least 200 Da, fatty acids having a molecular weight of at least 200 Da, and combinations thereof, wherein the adjuvant forms between about 0.01% and about 5% of the batter or dough and the oil forms between about 0.5% and about 50% of the batter or dough.

In certain embodiments, the oil is selected from the group consisting of liquid oils, semi-solid and solid fats, fat replacers, and combinations thereof in any proportion. In particular embodiments, the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil. In specific embodiments, the oil comprises an oil or fat selected from the group consisting of a partially or fully hydrogenated oil, an animal-derived fat, margarine, coconut oil, palm oil, palm kernel oil, butter fat, and cocoa fat. In certain embodiments, the oil comprises a fat replacer selected from the group consisting of diglycerides, triglycerides, combinations of short- and long-chain fatty acids, sucrose esters, and edible indigestible hydrophobic materials. In particular embodiments, the oil comprises at least about 25% of a fat replacer having a caloric value of less than 6.9 kcal/g.

In certain embodiments, the adjuvant comprises at least one fatty alcohol and at least one fatty acid in a ratio between about 2:8 and about 8:2. In particular embodiments, the fatty alcohol is stearyl alcohol and the fatty acid is behenic acid.

In some embodiments, the adjuvant comprises a fatty alcohol having between about 15 and about 50 carbon atoms in its hydrocarbon chain. In certain embodiments, the fatty alcohol is selected from the group consisting of cetyl alcohol, stearyl alcohol, arachidyl alcohol, behenyl alcohol (docosanol), octacosanol, and 1-triacontanol. In particular embodiments, the fatty alcohol has a branched hydrocarbon chain containing at least one alkyl group side chain. In specific embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In certain embodiments, the fatty alcohol has at least one additional hydroxyl group linked to its hydrocarbon chain. In particular embodiments, the fatty alcohol has at least one double bond in its hydrocarbon chain.

In some embodiments, the adjuvant comprises a fatty acid having between about 15 and about 50 carbon atoms in its hydrocarbon chain. In certain embodiments, the fatty acid is selected from the group consisting of hexadecanoic (palmitic) acid (C16), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30). In particular embodiments, the fatty acid has a branched hydrocarbon chain containing at least one alkyl group side chain. In specific embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In certain embodiments, the fatty acid has at least one additional hydroxyl group linked to its hydrocarbon chain. In particular embodiments, the fatty acid has at least one double bond in its hydrocarbon chain.

In some embodiments, the batter or dough further comprises at least one additive selected from the group consisting of emulsifiers, stabilizers, anti-oxidants, flavors, and colorants.

Another aspect of the invention provides a baked good comprising the batter or dough. In particular embodiments, the baked good is selected from the group consisting of cake, muffin, tart, bread, bun, bagel, cookie, biscuit, wafer, pie, pizza, pita, doughnut, pancake, cracker, and pastry. In some embodiments, the baked good has a reduced caloric value. In certain embodiments, the baked good has a cholesterol-lowering effect.

Still another aspect of the invention provides a baked good comprising an adjuvant selected from the group consisting of fatty alcohols having a molecular weight of at least 200 Da, fatty acids having a molecular weight of at least 200 Da, and combinations thereof. In some embodiments, the adjuvant forms between about 0.05% and about 5% of the baked good. In certain embodiments, the adjuvant comprises at least one fatty alcohol and at least one fatty acid in a ratio between about 2:8 and about 8:2. In particular embodiments, the fatty alcohol is stearyl alcohol and the fatty acid is behenic acid. In specific embodiments, the baked good is selected from the group consisting of cake, muffin, tart, bread, bun, bagel, cookie, biscuit, wafer, pie, pizza, pita, doughnut, pancake, cracker, and pastry.

In yet another aspect, the invention provides a baked good comprising an oil composition, said oil composition comprising a mixture of an oil and an adjuvant selected from the group consisting of fatty alcohols having a molecular weight of at least 200 Da, fatty acids having a molecular weight of at least 200 Da, and combinations thereof, wherein the adjuvant forms between about 0.01% and about 5% of the baked good and the oil forms between about 0.5% and about 50% of the baked good.

In certain embodiments, the oil is selected from the group consisting of liquid oils, semi-solid and solid fats, fat replacers, and combinations thereof in any proportion. In particular embodiments, the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil. In specific embodiments, the oil comprises an oil or fat selected from the group consisting of a partially or fully hydrogenated oil, an animal-derived fat, margarine, coconut oil, palm oil, palm kernel oil, butter fat, and cocoa fat. In certain embodiments, the oil comprises a fat replacer selected from the group consisting of diglycerides, triglycerides, combinations of short- and long-chain fatty acids, sucrose esters, and edible indigestible hydrophobic materials. In particular embodiments, the oil comprises at least about 25% of a fat replacer having a caloric value of less than 6.9 kcal/g.

In certain embodiments, the adjuvant comprises at least one fatty alcohol and at least one fatty acid in a ratio between about 2:8 and about 8:2. In particular embodiments, the fatty alcohol is stearyl alcohol and the fatty acid is behenic acid.

In some embodiments, the adjuvant comprises a fatty alcohol having between about 15 and about 50 carbon atoms in its hydrocarbon chain. In certain embodiments, the fatty alcohol is selected from the group consisting of cetyl alcohol, stearyl alcohol, arachidyl alcohol, behenyl alcohol (docosanol), octacosanol, and 1-triacontanol. In particular embodiments, the fatty alcohol has a branched hydrocarbon chain containing at least one alkyl group side chain. In specific embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In some embodiments, the fatty alcohol has at least one additional hydroxyl group linked to its hydrocarbon chain. In certain embodiments, the fatty alcohol has at least one double bond in its hydrocarbon chain.

In some embodiments, the adjuvant comprises a fatty acid having between about 15 and about 50 carbon atoms in its hydrocarbon chain. In certain embodiments, the fatty acid is selected from the group consisting of hexadecanoic (palmitic) acid (C16), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30). In particular embodiments, the fatty acid has a branched hydrocarbon chain containing at least one alkyl group side chain. In specific embodiments, the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. In some embodiments, the fatty acid has at least one additional hydroxyl group linked to its hydrocarbon chain. In certain embodiments, the fatty acid has at least one double bond in its hydrocarbon chain.

In some embodiments, the baked good further comprises at least one additive selected from the group consisting of emulsifiers, stabilizers, anti-oxidants, flavors, and colorants. In certain embodiments, the baked good is selected from the group consisting of cake, muffin, tart, bread, bun, bagel, cookie, biscuit, wafer, pie, pizza, pita, doughnut, pancake, cracker, and pastry.

In particular embodiments, the baked good has a reduced caloric value. In specific embodiments, the baked good has a cholesterol-lowering effect.

Another aspect of the invention provides a method of reducing or preventing an increase in a blood cholesterol level comprising administering to a subject in need thereof an effective amount of the baked good. Still another aspect provides a method of treating or preventing hyperlipidemia or hypercholesterolemia comprising administering to a subject in need thereof an effective amount of the baked good. Yet another aspect provides a method of promoting weight loss or preventing weight gain comprising administering to a subject in need thereof an effective amount of the baked good.

In a further aspect, the invention provides a method of making a batter or dough. The method comprises combining an oil with an adjuvant selected from the group consisting of fatty alcohols having a molecular weight of at least 200 Da, fatty acids having a molecular weight of at least 200 Da, and combinations thereof, thereby forming an oil composition, and combining the oil composition with additional baking ingredients, thereby forming a batter or dough. In some embodiments, combining the adjuvant with the oil comprises heating the oil above the melting temperature of the adjuvant, adding the adjuvant to the heated oil, and mixing the adjuvant with the oil. In certain embodiments, combining the adjuvant with the oil comprises mixing the adjuvant with a portion of the oil, thereby forming an adjuvant-oil concentrate, and mixing the adjuvant-oil concentrate with the remainder of the oil, thereby forming the oil composition. In some embodiments, the method further comprises combining a fatty alcohol a molecular weight of at least 200 Da and a fatty acid having a molecular weight of at least 200 Da in a ratio of between about 2:8 and about 8:2, thereby forming the adjuvant. In particular embodiments, of the method, the oil composition comprises between about 70% and about 99.95% of the oil and between about 0.05% and about 30% of the adjuvant.

Another aspect of the invention provides a method of making a baked good. The method comprises combining an oil with an adjuvant selected from the group consisting of fatty alcohols having a molecular weight of at least 200 Da, fatty acids having a molecular weight of at least 200 Da, and combinations thereof, thereby forming an oil composition, combining the oil composition with additional baking ingredients, thereby forming a batter or dough, and baking the batter or dough, thereby forming a baked good. In some embodiments, combining the adjuvant with the oil comprises heating the oil above the melting temperature of the adjuvant, adding the adjuvant to the heated oil, and mixing the adjuvant with the oil. In certain embodiments, combining the adjuvant with the oil comprises mixing the adjuvant with a portion of the oil, thereby forming an adjuvant-oil concentrate, and mixing the adjuvant-oil concentrate with the remainder of the oil, thereby forming the oil composition. In particular embodiments, the method further comprises combining a fatty alcohol having a molecular weight of at least 200 Da and a fatty acid having a molecular weight of at least 200 Da in a ratio of between about 2:8 and about 8:2, thereby forming the adjuvant. In certain embodiments of the method, the oil composition comprises between about 70% and about 99.95% of the oil and between about 0.05% and about 30% of the adjuvant. In particular embodiments of the method, the baked good has reduced caloric value.

### BRIEF DESCRIPTION OF THE DRAWING

The invention is described with reference to the following Figures, which are for the purpose of illustration and not limiting of the invention.

FIG. 1 is a photograph of cakes, sample numbers 1-4, according to certain embodiments of the invention.

FIG. 2 is a photograph of cakes, sample numbers 5-8, according to certain embodiments the invention.

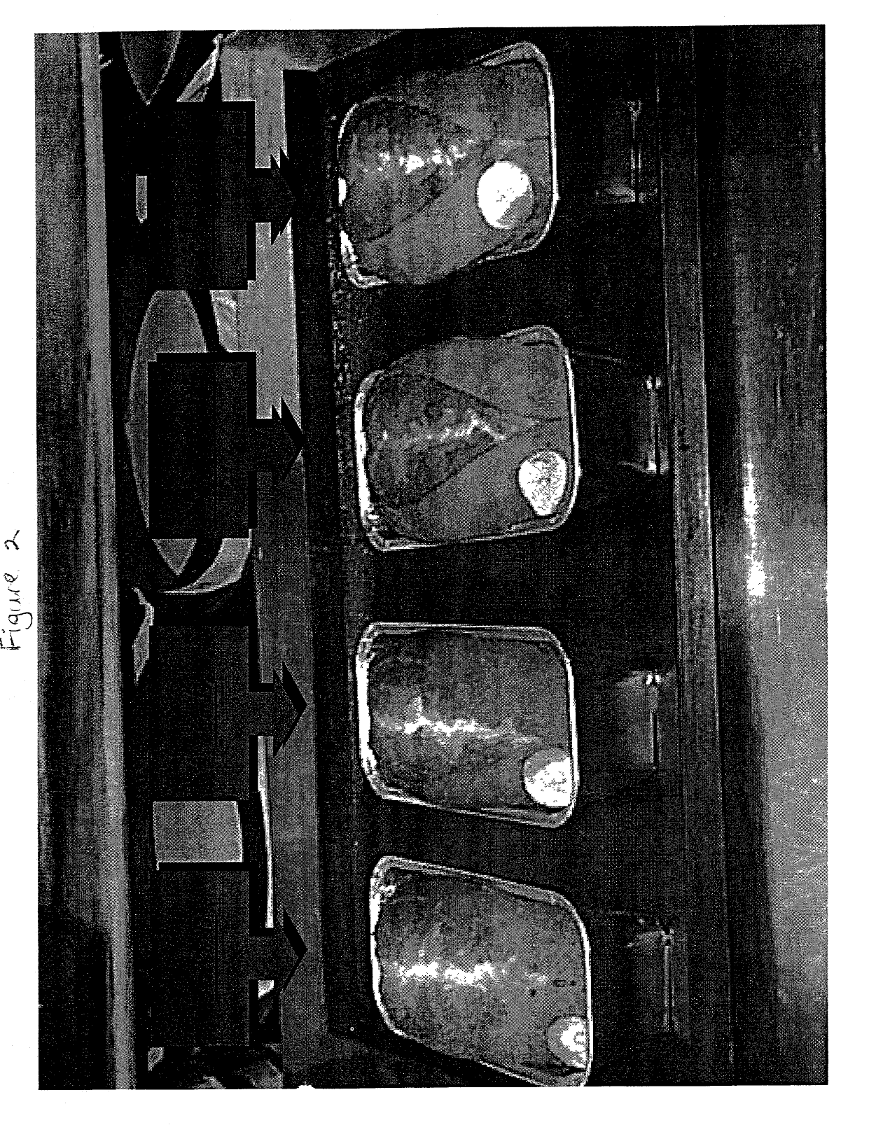

### DETAILED DESCRIPTION

One aspect of the invention provides an oil composition including a mixture of an edible oil and a baking adjuvant, and optionally one or more edible additives. The oil composition is useful for creating baked goods, such as cakes, muffins, breads, pastries, biscuits, shortbreads, shortcakes, and the like, that have improved texture and higher volume compared to similar baked goods made using the same recipe but with conventional oils and shortenings. Particularly useful oil compositions include fat replacers and/or oils rich in unsaturated fat and low in saturated fat. Such oil compositions are healthier than commonly used fat, margarine, and shortenings, which contain large amounts of unhealthy saturated and trans fats. Such oil compositions are useful for producing baked goods having reduced caloric value and baked goods having a cholesterol-lowering effect. “Reduced caloric value” means that the calorie content of the baked good made with the oil composition as described herein is less than that of a comparable baked good made using conventional shortening. “Cholesterol-lowering effect” refers to a reduction or lack of increase in the blood cholesterol level of a subject that is caused or aided by the subject's eating baked goods including oil compositions described herein, particularly when such baked goods are eaten in place of conventional baked goods that are rich in unhealthy saturated and trans fat.

Baking Adjuvants

The oil composition includes a mixture of at least one oil and at least one baking adjuvant. The baking adjuvant thickens the oil, providing a consistency suitable for use in baking. The baking adjuvant also allows for production of a batter or dough having one or more enhanced properties such as improved mixing capability, smoother, more homogeneous texture, increased aeration, and/or improved health profile, in comparison to similar batters and doughs produced using conventional oils, fats, or shortenings. Further, the baking adjuvant allows for production of baked goods having one or more enhanced properties such as increased volume, improved texture, and/or improved health profile, in comparison to similar baked goods produced using conventional oils, fats, or shortenings. An “improved health profile” refers to one or more of a reduced caloric content, a reduced content of saturated and/or trans fat, and/or an increased content of unsaturated fat. In some embodiments, the baking adjuvant (also referred to herein as “adjuvant”) includes one or more edible fatty acids and/or edible fatty alcohols. “Edible,” as used herein, means acceptable for human consumption. Particularly useful adjuvants include one or more fatty acids and/or fatty alcohols having a molecular weight of at least 200 Da.

Suitable edible fatty acids include fatty acids having 15 or more carbon atoms in their hydrocarbon chain. In certain embodiments, the fatty acids have 17 or more carbon atoms in their hydrocarbon chain. Non-limiting examples include heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), as well as fatty acids with longer hydrocarbon chains (up to C50), and mixtures thereof, which are acceptable for human ingestion.

Suitable edible fatty alcohols, also referred to in the art as monomeric fatty alcohols, include fatty alcohols having 15 or more carbon atoms in their hydrocarbon chain, e.g., cetyl alcohol (C16), stearyl alcohol (C18), and mixtures thereof, that are acceptable for human ingestion (see U.S. Food and Drug Administration, Center for Food Safety & Applied Nutrition, EAFUS: A Food Additive Database). Further non-limiting examples of suitable fatty alcohols include arachidyl alcohol (C20), behenyl alcohol (docosanol) (C22), octacosanol (C28), 1-triacontanol (C30), as well as alcohols with longer hydrocarbon chains (up to C50).

Other useful adjuvants include fatty acids and/or fatty alcohols having a molecular weight of at least 200 Da and at least one double bond in their hydrocarbon chain. Still other useful adjuvants include fatty acids and/or fatty alcohols having molecular weight of at least 200 Da, wherein the hydrocarbon chain is branched, containing at least one alkyl group side chain. Non-limiting examples of alkyl group side chains include methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups. Yet another suitable class of adjuvants includes fatty acids and/or fatty alcohols having at least one additional hydroxyl group linked to their hydrocarbon chain.

Particularly useful adjuvants include fatty alcohols having 16 to 20 carbon atoms in their hydrocarbon chain, with no double bonds or alkyl group side chains, e.g., cetyl alcohol, stearyl alcohol, and arachidyl alcohol, and fatty acids having 18 to 22 carbon atoms in their hydrocarbon chain, with no double bonds or alkyl group side chains, e.g., stearic acid, arachidic acid, and behenic acid. In specific embodiments, a combination of a fatty acid and a fatty alcohol in a ratio between about 2:8 and about 8:2 is used. A non-limiting example of such an adjuvant is a 1:1 mixture of stearyl alcohol and behenic acid.

Another class of baking adjuvant includes mixtures of fatty alcohols and/or fatty acids contained in or derived from natural waxes, such as beeswax, in which a majority of the fatty alcohols and/or fatty acids have at least 18 carbon atoms in their hydrocarbon chain.

Oils

Oil compositions are provided. The term “oil” is used herein to encompass oils, semi-solid and solid fats, and “fat replacers,” as defined below. Accordingly, an “oil composition” as described herein contains one or more oils, fats, fat replacers, or combination thereof in any proportion.

The fats or oils suitable for use herein can be fluid or plastic. Fluid oils have a high content of unsaturated fatty acids, while plastic fats have a higher content of saturated fatty acids. Suitable liquid oils include oils of vegetable or marine origin. Non-limiting examples include olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, castor oil, linen oil, matthiola incana oil, garlic oil, shark oil, hypericum oil, sweet basil oil, and mixtures thereof at any proportion. Particularly useful oils include poly-unsaturated oils containing omega-3 and omega-6 fatty acids. For example, in certain embodiments, the unsaturated oil contains at least about 6% of omega-3 oil, omega-6 oil, or a mixture thereof. All percentages set forth herein are by weight unless defined otherwise.

In some embodiments, even oils that are solid or semi-solid at ambient conditions, due to the high degree of saturation of their fatty acids, are improved by the addition of adjuvants. Such oils may be derived from plant or animal sources. Non-limiting examples of such semi-solid or solid oils include partially or fully hydrogenated vegetable oil, marine oil, coconut oil, palm oil, palm kernel oil, butter oil, tallow, lard, milk fat, and chicken fat.

Another useful class of oils and fats includes fat replacers. The term “fat replacer,” as used herein, refers to edible fatty substances that are produced artificially in order to achieve a fatty material that is functionally compatible with conventional fats and has therapeutic benefits due to its chemical structure and its particular metabolic pathways. The production of such fat replacers may involve chemical synthesis or enzymatic processing. One common property of fat replacers is that their caloric value is less than about 6.9 kcal/g. Fat replacers are also referred to as “fat substitutes” and “synthetic fats.” Fat replacers are classified by their chemical nature, as exemplified below.

Monoacylglycerols and diacylglycerols have long been used as emulsifiers and contribute sensory properties comparable to fats. By using specific fatty acids in the formulation of these compounds, it is possible to achieve desired functionality at reduced energy (e.g., 5 kcal/g vs. 9 kcal/g for conventional fats).

Further fat replacers include long-chain diacylglycerols, mainly comprising unsaturated and/or saturated fatty acids having at least 16 carbon atoms in their hydrocarbon backbone. In some embodiments, the fatty acids comprised by such long-chain diacylglycerols include 55% or more, and in particular embodiments 70% or more, of unsaturated fatty acids. Specific embodiments include between about 20% and about 65% of oleic acid, and between about 15% and about 65% of linoleic acid. Such diacylglycerols typically include greater than about 80% diacylglycerol as a mixture of 1,3-diglyceride and 1,2-diglyceride at a ratio of about 7:3. This composition of fat is readily hydrolyzed to monoglycerides and fatty acids in the gastrointestinal tract. The main metabolic product is 1-monoglyceride, which is further hydrolyzed into free fatty acids and glycerol, while the minor product 2-monoglyceride is re-esterified into triglycerides. Furthermore, such diacylglycerols are believed to aid in the maintenance or loss of weight and fat mass, and help to maintain healthy triglyceride levels in the bloodstream. Another class of useful diacylglycerols includes medium-chain unsaturated and/or saturated fatty acids having between about 8 and about 12 carbon atoms in their carbon atom backbone. Mixed diacylglycerols having both long-chain fatty acids and medium-chain fatty acids are also useful.

Triacylglycerols containing selected short- and long-chain fatty acids can also provide the sensory characteristics of fat with reduced energy content because they are not efficiently absorbed. Salatrim (acronym for short- and long-chain acyl triglyceride molecules), a family of structured triglycerides prepared with a combination short- and long-chain fatty acids, is representative of this class. Such a fat replacer is useful in a wide variety of food applications, because it affords the physical properties of fat with reduced calorie (5 kcal/g vs. 9 kcal/g for conventional oil) and fat content. It can replace conventional oils in many foods such as confections, spreads, baked goods, and snacks. However, since it is liquid, its uses are limited, as many food products require solid or semi-solid fat to achieve desirable texture and taste.

Medium-chain triglycerides have long been recognized for their therapeutic potential. Medium-chain triglycerides, having fatty acids with between about 6 and about 12 carbon atoms in their carbon chain backbone, are metabolized differently than long-chain triglycerides (C14-C24). Long-chain triglycerides are hydrolyzed, then re-esterified to triglycerides, then imported into chylomicrons, which enter the lymphatic system. Medium-chain triglycerides bypass the lymphatic system. They are hydrolyzed to medium-chain fatty acids, which are transported via the portal vein directly to the liver, where they are oxidized for energy. They are not likely to be stored in adipose tissue. For enteral and parenteral feeding, their advantage is already known. Caprocaprylobehenic triacylglyceride, commonly known as caprenin, is manufactured from glycerol by esterification with caprylic (C8), capric (C10), and behenic (C22) fatty acids. Because behenic acid is only partially absorbed, and capric and caprylic acids are more readily metabolized than other longer chain fatty acids, caprenin provides only 5 kcal/g.

Another type of fat replacer binds fatty acids to nontraditional backbones (e.g., sugar), so that enzymes in the human gut are not able to cleave the fatty acids. Thus, they are not absorbed and do not contribute energy. Advantageously, such compounds are heat stable and retain their functional properties in baked and fried foods. An example of this class of compounds is olestra. Olestra is a mixture of hexa-, hepta-, and predominantly octa-esters of sucrose, which are not hydrolysed in the intestine and are not absorbed. They are formed by reaction between sucrose and fatty acid esters obtained from edible fats and oils (soybean, maize, coconut, and cottonseed) of carbon chain length C8-C22, and include unsaponifiable materials (0.08%-0.3%) present in these fats and oils. Olestra has properties similar to those of a naturally occurring fat. However, unlike the natural products, olestra provides no calories or saturated fat because it is indigestible. It passes through the digestive tract but is not absorbed into the body.

Another member of this group is orbestrin, a low-calorie, heat stable, liquid fat substitute composed of fatty acid esters of sorbitol and sorbitol anhydrides. Orbestrin has approximately 1.5 kcal/gram.

Any oil, fat, fat replacer, or combination thereof in any proportion may be used in the oil compositions described herein.

Oil Compositions

In certain embodiments, an oil composition is provided including one or more oils, fats, and/or fat replacers, and one or more adjuvants, as described above. The beneficial effects of the adjuvant in the oil composition are concentration-related. Higher concentrations contribute more to the texture and volume of the baked product. In certain embodiments, the total concentration of the adjuvant in the oil composition is between about 0.1% and about 18% of the oil or fat mass. In particular embodiments, concentrations between about 0.5% and about 10% are used. In specific embodiments, the adjuvant concentration is between about 0.5% and about 7.5%. Incorporation of adjuvants in such concentrations affords baked goods having improved properties and an excellent organoleptic profile.

The oil composition is prepared by mixing an oil with a baking adjuvant, which thickens the oil. In certain embodiments, mixing the adjuvant with the oil produces an oil composition having a lard-like consistency suitable for use in baking. In some embodiments, an oil composition is produced by warming up an oil or fat to a temperature above the melting temperature of an adjuvant (typically about 50° C. to about 80° C.), adding the adjuvant in a solid or melted form, and mixing until a clear solution is obtained. In specific embodiments, the mixture is then cooled. Another method of preparing the oil or fat composition is to pump the oil and the adjuvant simultaneously through a low-volume mixing chamber equipped with a heating system and into a receiving tank.

In some embodiments, a small portion of the oil (typically between about 5% and about 50% of the total oil mass) is warmed and mixed with the adjuvant as described above to make an adjuvant-oil concentrate. The adjuvant-oil concentrate is added to the remainder of the oil mass to obtain the final desired concentration. In alternative embodiments, the adjuvant-oil concentrate is presented as a stand-alone product, which is supplied to a bakery for addition to baking mixtures. The remaining quantity of oil required for producing a baked good is added separately and mixed together with the rest of the baking ingredients. In still other embodiments, the adjuvant-oil concentrate or the oil composition is provided as part of a baked good mix or baking kit that includes the adjuvant-oil concentrate or oil composition and some or all of the additional ingredients for making one or more baked goods. For example, a dry bake mix is provided including an adjuvant-oil concentrate or oil composition along with the remaining dry (i.e., non-liquid) ingredients required for baking a particular baked good such as, for example, a cake.

In certain embodiments, especially in the production of dough with high fat content, an oil composition as described herein is presented as part of a margarine-like emulsion containing between about 40% and about 90% of the oil composition and between about 10% and about 60% water. Optional additional components of the margarine-like emulsion include, but are not limited to, emulsifiers, stabilizers, flavors, and colorants.

Oil or fat compositions as described herein optionally include food additives. Such additives are well-known in the art. Non-limiting examples include emulsifiers, stabilizers, anti-oxidants, flavors, colorants, and other food ingredients used in the baking industry.

The oil or fat compositions can be used in the production of baked goods immediately after preparation, or can be stored and used at a later stage.

Batter, Dough, and Baking Kit

In certain embodiments, a batter or dough is prepared with an oil composition as described above. The oil composition is prepared and then combined with the additional baking ingredients required for making a particular baked good to form the batter or dough. Additional baking ingredients encompass any conventional baking ingredients. Such ingredients are well-known in the art. The ingredients of baked goods generally include flour, oil and/or fat, water or milk, some flavoring substance such as, for example, salt, and some coloring substance, such as, for example, milk solids or corn sugars. Flours suitable for use in the doughs and baked goods described herein include, but are not limited to, any commonly used flour for pastry, biscuit, shortcake, or shortbread dough. Further non-limiting examples of suitable flours include all-purpose flour, unbleached pastry flour, bread flour, and cake flour. One of skill in the art will understand that the amount of water and/or milk in the dough can vary. Higher or lower moisture levels result in doughs which are easy or difficult to handle and which form into desired shapes. Non-limiting examples of additional optional ingredients in batters and doughs for making baked goods include baking powder, eggs, and sugar. In the case of cakes, cookies, bread, wafers, and the like, the ingredient systems tend to incorporate relatively high levels of sugar (greater than about 20%) and/or leavening agents, emulsifiers, and the like, in addition to flour, fat, water or milk, and flavoring and coloring agents. These additional ingredients generally have an impact on texture, making the baked goods aerated, or chewy, or cakey, etc.

Various other additives can be included in the fat phase of the batter or dough. In certain embodiments, the fat or oil composition includes one or more additives. Non-limiting examples of suitable additives include flavorings, emulsifiers such as, for example, sucrose esters, mono- and diglycerides, and lactylated glycerides, anti-spattering agents, anti-sticking agents, anti-oxidants, and the like. As with standard shortenings, nitrogen also can be added to the fat product during processing to improve the lightness or color of the product. In some embodiments, the fat part of the product is fortified with vitamins or minerals, particularly the fat-soluble vitamins. The fat-soluble vitamins include vitamin A, vitamin D, vitamin K, and vitamin E (tocopherol or tocopherol esters).

Artificial sweeteners optionally can be added to the dough compositions, alone or in combination with bulking agents, to provide an extra caloric reduction benefit. Noncaloric or reduced calorie sweeteners include, but are not limited to, aspartame, saccharin, alitame, thaumatin, dihydrochalcones, cyclamates, steviosides, glycyrrhizins, and synthetic alkoxy aromatics. Edible preservatives also can be included in the dough compositions in amounts ranging from about 0.03% to about 0.3%. Suitable edible preservatives include, but are not limited to, calcium propionate, propionic acid, sorbic acid, potassium sorbate, and sodium benzoate.

In certain embodiments, an adjuvant-oil concentrate or oil composition as described above is provided as part of a baked good mix or baking kit that also includes some or all of the additional ingredients for making a batter or dough to produce one or more baked goods. The additional ingredients include any combination of the conventional baking ingredients, optional additives, artificial sweeteners, preservatives, etc. described above. The baked good kit or baking mix is used to prepare a dough or batter for making a baked good. For example, in one embodiment, a dry bake mix is provided including an oil composition as described herein along with the remaining dry (i.e., non-liquid) ingredients required for baking a particular baked good such as, for example, a cake. The oil composition and dry ingredients in the bake mix are combined with the necessary liquid ingredients for making a cake to produce a cake batter.

In at least some embodiments, the batter or dough described herein is more aerated and smooth than conventional batters or doughs, and can produce a baked product with improved texture and/or increased volume. It was found that a batter or dough including an oil composition as described herein requires less mixing time in order to become smooth and homogeneous in comparison with a batter or dough prepared with the same oil but without the adjuvant. Furthermore, it was also found that in many cases including an oil composition as described herein allows for mixing all of the ingredients at once, rather than adding them stepwise, as is customary in the baking industry. Thus, such a batter or dough allows for energy cost savings. Moreover, it was found that the degree of aeration, as measured by the specific gravity of the batter or dough after mixing, is greater when using an oil composition as described herein in comparison with the same batter or dough prepared without the adjuvant. The extent of improvement is positively correlated to the portion of oil in the dough mass. Likewise, the specific gravity of the batter or dough is inversely correlated with the amount of oil in the batter or dough, i.e., the more oil in the batter or dough, the more aerated the dough is. Thus, baked products having a high percentage of oil are more influenced by the adjuvants than baked products containing low amounts of oil. In certain embodiments, the batter or dough includes between about 0.5% and about 50% oil, sometimes between about 2% and about 30% oil. In some embodiments, such batters or doughs contain between about 0.005% and about 5% of an adjuvant.

Batters and doughs including an oil composition as described herein can be used for baking right after their preparation. They also can be refrigerated or frozen, for baking after storage. Such batters and doughs are useful and beneficial in all sorts of baked goods, including, without limitation, cake, muffin, tart, bread, bun, bagel, cookie, biscuit, wafer, pie, pizza, pita, doughnut, pancake, cracker, and pastry. In particular embodiments, the batter or dough including an oil composition is used to prepare a cake, muffin, bread, bun, or any other baked product having a soft texture in at least part of its body. The oil compositions described herein afford baking doughs that produce baked goods with improved qualities compared to baked goods made from doughs produced with conventional oils or fats. The improved qualities include enhanced volume and improved texture.

For example, when cakes are produced using the oil compositions described herein, their volume is enhanced, affording a cake with up to about 30% more volume compared to a similar cake made with regular oils or semi-solid fats. The effect on the volume positively correlates with the concentration of adjuvant in the oil. In certain embodiments, an adjuvant concentration between about 0.75% and about 12% is used. In further embodiments, an adjuvant concentration between about 2% and about 7.5% is used. The volume further correlates with the proportion of oil in the batter—the more oil in the batter, the more pronounced the volume enhancement. Thus, this volume enhancement property is particularly important in the production of cakes and muffins having between about 5% and about 30% oil in their batter. It also has been found that the increase in volume can be controlled by decreasing the amount of emulsifiers and/or stabilizers in the baking mixture, and by applying milder mixing, such that the cost of ingredients is lower and the production of baked goods is more efficient and economical.

The effect of the adjuvant on the texture of the baked good is pronounced even in baked goods with low oil content, such as bread. In bread, the grains are more even when an oil composition as described herein is employed instead of a conventional oil or semi-solid fat.

An oil composition as described herein also provides a baked product that is healthier than the equivalent product made using conventional fats. It is known in the art of baking that the fats commonly used in baking are semi-solid and solid fats, which are substantially saturated and commonly contain trans fatty acids. Such fats are known to cause undesirable effects on the cardiovascular system, as discussed in the background section above. By contrast, liquid vegetable oils, which are substantially unsaturated or polyunsaturated and have cardiovascular protective effects, cannot usually be used in baking. As demonstrated in the examples below, use of the adjuvants described herein enables the usage of liquid vegetable oils in baking, and thus allows delivery of cardioprotective benefits in the baked products. This is particularly pronounced when oils rich in monounsaturated fatty acids, such as oleic acid and/or omega-3 polyunsaturated fatty acids, which are known to reduce blood LDL cholesterol level, are used in the oil composition. An oil composition as described herein also enables delivery of health benefits using baked goods as a delivery system when fat replacers having both low caloric value and cholesterol-lowering properties are used as part of the oil composition.

The adjuvants described herein also afford health benefits. Long-chain fatty alcohols have been documented to reduce serum cholesterol levels in experimental models, healthy humans, and hypercholesterolemic patients. Such alcohols have been employed in the treatment of elevated serum cholesterol levels, as reported in a number of published human clinical trials (see, e.g., Pons, et al., Curr. Ther. Res. 52:507-513 (1992)). Fatty acids having at least 18 carbon atoms in their backbone have recently been shown to improve thrombogenic and atherogenic risk factor profiles in humans (Kelly et al., Eur. J. Clin. Nutr. 55:88 (2001)). When both the oil and the adjuvant of the oil composition reduce serum cholesterol levels and afford other metabolic benefits, such a mixture may afford a synergistic beneficial effect.

In certain embodiments, oil compositions including a cholesterol-lowering oil or fat and an adjuvant, and baked goods produced therefrom, are used to deliver a therapeutic effect to patients with metabolic disorders, such as hypercholesterolemia, hyperlipidemia, diabetes, syndrome X, and other syndromes involving or resulting from high levels of blood cholesterol and lipids. In particular embodiments, the oil compositions and baked goods are used as prophylaxis in subjects who are at risk of developing such metabolic disorders and syndromes. In other embodiments, an oil composition described herein is used to produce a baked good with reduced caloric value. Such baked goods are most beneficial for subjects who require low-caloric diets for weight control.

Certain embodiments of the invention are illustrated by the following examples, which are not intended to be limiting of the invention.

### EXAMPLE 1

### Yellow Cake

This example provides guidelines for the development of new recipes using a liquid oil with a baking adjuvant instead of a conventional shortening. This example demonstrates the ability to control the specific gravity of a batter using a liquid oil and a baking adjuvant. In summary, the use of a baking adjuvant clearly affects the specific gravity of the batter. Inclusion of a baking adjuvant in an amount as low as 0.25% of the batter (2% of the oil) produced beneficial effects. Distilled monoglyceride emulsifiers (without a baking adjuvant) had a very limited effect on batter specific gravity when present in an amount of 0.25% of a batter including liquid oil. The use of a baking adjuvant together with a distilled monoglyceride produced a synergistic effect. The amount of emulsifier required was as low as 0.25% of the batter, which is much lower than the amount traditionally required. Gluten protein was not needed to reduce specific gravity when a baking adjuvant was used. Changing the percentage of egg powder did not affect the reduction in specific gravity caused by the baking adjuvant.

Composition

| | % |
|---|---|
| Sugar | 26% |
| Egg powder | 5.5%* |
| Skim milk powder | 1.5% |
| Tap water | 23% |
| White flour | 30% |
| Baking powder | 0.75% |
| Salt | 0.35% |
| Fat or oil, selected from the following: | 13% |
| a) Emulsified shortening | |
| b) Liquid oil | |
| c) Liquid oil with distilled monoglyceride | |
| d) Liquid oil + Baking adjuvant | |
| e) Liquid oil + Baking adjuvant + Monoglyceride | |
| Total | 100% |

*Replaced by sugar in recipe with reduced egg powder

Method

The sugar, egg powder, skim milk powder and water were added to a KitchenAid® mixer bowl and mixed using a flat beater to produce a smooth liquid. The baking adjuvant was mixed with 20% of the liquid oil at 80° C. and cooled to room temperature, and then the rest of the oil was added and mixed together. (Alternatively, the baking adjuvant was mixed with the full mass of oil at 80° C. and cooled down to room temperature). The oil+baking adjuvant was poured into the mixer bowl and the flour with the baking powder and salt were added. The mixture was stirred at low speed for 2 minutes, scraped down, and mixed again at medium speed for 2 minutes. The specific gravity of the batter was then measured.

Results

| | Baking Adjuvant | Baking Adjuvant | | | | |
|---|---|---|---|---|---|---|
| | (1:1 mixture of | (1:1 mixture of | Distilled | | | |
| | stearyl alcohol + | stearyl alcohol + | Mono- | | Whole Egg | Specific |
| Exp. | behenic acid) | behenic acid) | glyceride | Flour | Powder | Gravity |
| No. | % in Oil | % of Batter | % of Batter | Type | % of Batter | of Batter |
| 1 | emulsified shortening | | positive | White | 5.5 | 0.95 |
| 2 | 0 | 0 | 0 | wheat | | 1.12 |
| 3 | 0 | 0 | 0.25 | | | 1.12 |
| 4 | 0 | 0 | 0.5 | | | 1.12 |
| 5 | 0 | 0 | 1 | | | 0.97 |
| 6 | 1 + 1 | 0.25 | 0 | | | 0.90 |
| 7 | 1 + 1 | 0.25 | 0.25 | | | 0.65 |
| 8 | 1 + 1 | 0.25 | 0.5 | | | 0.66 |
| 9 | 1 + 1 | 0.25 | 1 | | | 0.68 |
| 10 | 1.5 + 1.5 | 0.38 | 0 | | | 0.87 |
| 11 | 2 + 2 | 0.5 | 0 | | | 0.83 |
| 12 | 1.5 + 1.5 | 0.38 | 0.5 | | | **0.55 |
| 13 | 1.5 + 1.5 | 0.38 | 1 | | | **0.54 |
| 14 | 1.5 + 1.5 | 0.38 | 0.25 | | | 0.66 |
| 15 | 0 | 0 | 0 | Wheat | | 1.14 |
| 16 | 1.5 + 1.5 | 0.38 | 0 | gluten free | | 0.87 |
| 17 | 1.5 + 1.5 | 0.38 | 0.25 | | | 0.73 |
| 18 | 0 | 0 | 0 | Corn | | 1.16 |
| 19 | 1.5 + 1.5 | 0.38 | 0 | Starch | | 0.87 |
| 20 | 1.15 + 1.5 | 0.38 | 0.25 | | | 0.72 |
| 21 | 0 | 0 | 0 | Potato | | 1.12 |
| 22 | 1.5 + 1.5 | 0.38 | 0 | Starch | | 0.85 |
| 23 | 1.5 + 1.5 | 0.38 | 0.25 | | | 0.64 |
| 24 | 0 | 0 | 0 | White | 2.75 | 1.12 |
| 25 | 0 | 0 | 0 | wheat | 3.75 | 1.12 |
| 26 | 1.5 + 1.5 | 0.38 | 0 | | 2.75 | 0.9 |
| 27 | 1.5 + 1.5 | 0.38 | 0 | | 3.75 | 0.88 |

**Adequate for sponge cake

Experiment (Exp. No.) 1 was the “control,” which was made with emulsified shortening. This experiment demonstrates the traditional way to produce a cake using fat containing saturated and trans fatty acids. Experiments 2-5 show that various emulsifier concentrations (up to 1%) have very little effect on the batter specific gravity when using liquid oil. Experiments 6-9 demonstrate that the baking adjuvant reduces the specific gravity of batter. Even when present at a level of 0.25% of the batter, the baking adjuvant caused a reduction of 20% in the specific gravity. In contrast, the same level of emulsifier had no effect on specific gravity. The baking adjuvant also showed a strong synergistic effect with a small level of emulsifier, causing an additional reduction of 25% in the batter specific gravity. Experiments 6-14 were designed to assess the level of synergistic effect using varying amounts of baking adjuvant and emulsifiers. It was found that the synergistic effect was significant at a low level of emulsifier (0.25% of the batter), while more emulsifier did not cause additional effects. Hence, using low levels of baking adjuvants enables a reduction in the amount of emulsifiers used, thus reducing the total cost of ingredients. Experiments 15-23 show that the baking adjuvant's effect on specific gravity is not connected to gluten protein, which is not present in the flours used in these experiments. This is probably due to reaction of the baking adjuvant with starch. Experiments 24-27 show that lowering the amount of egg powder in the batter did not affect the influence of the baking adjuvant on specific gravity. In terms of dietary value, the adjuvant-oil composition is advantageous, since the final product has a high content of unsaturated fatty acids, whereas products made with conventional shortenings contain high amounts of saturated and trans fatty acids, which are known to have untoward cardiovascular effects.

### EXAMPLE 2

### Madeira Cake with Liquid Oil

Composition

| | Grams |
|---|---|
| German flour | 180 |
| Corn starch | 20 |
| Sugar | 160 |
| Baking powder | 8 |
| Xanthan | 0.4 |
| Carboxy methyl cellulose | 0.4 |
| Salt | 1.2 |
| Spongolit 4040 | 40 |
| Oil composition | 128 |
| Egg | 160 |
| Water | 60 |

Oil Compositions

| | |
|---|---|
| Control No. 1 = | reference liquid oil (Sunflower or soybean oil) |
| Control No. 2 = | margarine |
| Oil Composition 3 = | 3.5% stearyl alcohol in liquid oil |
| Oil Composition 4 = | 7% stearyl alcohol in liquid oil |
| Oil Composition 5 = | 3.5% behenic acid in liquid oil |
| Oil Composition 6 = | 7% behenic acid in liquid oil |
| Oil Composition 7 | 3.5% stearyl alcohol/behenic acid |
| | blend (1:1) in liquid oil |
| Oil Composition 8 = | 7% stearyl alcohol/behenic acid |
| | blend (1:1) in liquid oil |

All oil compositions were used at ambient temperature.

Method

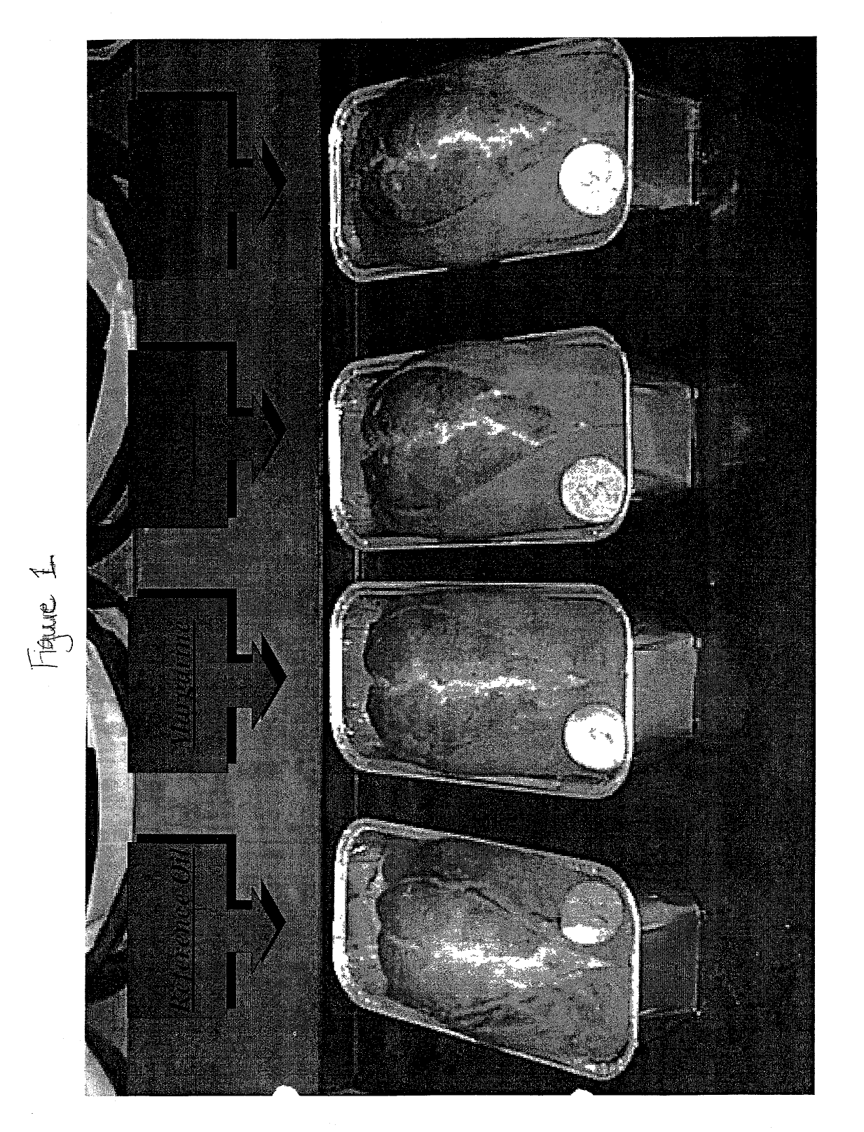

All cake constituents were mixed together and the cake was baked at 190° C. for 45 minutes. The resulting baked goods are shown in FIG. 1.

Results

A volume increase was observed in all adjuvant-containing samples (oil compositions 3-8). The greatest volume was obtained with oil compositions 7 and 8 (blend of stearyl alcohol/behenic acid). All products made with adjuvant had a finer pore structure. There was no difference in the pore structure observed between samples containing oil compositions 3-8. The dietary value of the adjuvant-oil composition is advantageous, since the final product has a high content of unsaturated fatty acids. In contrast, products made using traditional shortenings contain high amounts of saturated and trans fatty acids, which are known to have untoward cardiovascular effects.

### EXAMPLE 3

### Madeira Cake with Semi-solid Fat (Palm Oil)

Composition

The composition and procedure were identical to those described in Example 2, but oil compositions were based on palm oil, and palm oil without an adjuvant was used as a control.

Oil Compositions

| | |
|---|---|
| Oil Composition 1 = | 3.5% stearyl alcohol in palm oil |
| Oil Composition 2 = | 3.5% behenic acid in palm oil |
| Oil Composition 3 = | 3.5% stearyl/behenic (1:1) in palm oil |
| Control No. 1 = | Palm oil |

Results

A volume increase was observed for oil compositions 1, 2 and 3. The greatest volume volume was obtained with oil composition 3 (blend of stearyl alcohol and behenic acid). All products produced with adjuvant had a finer pore structure. No difference was observed between oil compositions 1, 2, and 3. This example demonstrates the enhancing effect of the baking adjuvant in a cake using a semi-solid fat.

### EXAMPLE 4

### White Bread

Composition

| | grams |
|---|---|
| Yeast | 40 |
| Water | 520 |
| Sugar | 20 |
| Skin milk powder | 10 |
| Improver BR - 15 | 20 |
| Oil composition | 96 |

Oil Composition

| | |
|---|---|
| Control No. 1 = | reference liquid oil (soybean or sunflower oil) |
| Control No. 2 = | margarine |
| Oil Composition 3 = | 3.5% stearyl alcohol in liquid oil |
| Oil Composition 4 = | 7% stearyl alcohol in liquid oil |
| Oil Composition 5 = | 3.5% behenic acid in liquid oil |
| Oil Composition 6 = | 7% behenic acid in liquid oil |
| Oil Composition 7 = | 3.5% stearyl/behenic blend (1:1) in liquid oil |
| Oil Composition 8 = | 7% stearyl/behenic blend (1:1) in liquid oil |

Production Conditions

| | |
|---|---|
| Mixing-time slow/fast | 6/1 |
| Dough-temp | Ambient |
| Dough rest | 20 min. |
| Dough weight | 500 gr. |
| Fermentation | 10 min. |

Results

A volume increase (2-4%) was observed in products containing an adjuvant. Low adjuvant effect was observed due to the very low fat content in the product. All products made with adjuvant had a finer pore structure and lighter color. Products made from oil compositions 7 and 8 had a very fine appearance.

### EXAMPLE 5

### Muffin

This example characterizes the concentration range of the baking adjuvant in order to obtain a favorable batter for a particular baked good. The example demonstrates the effect of the baking adjuvants on batter properties, and demonstrates the superiority of the adjuvants over other options.

Composition

| | % |
|---|---|
| White flour | 25.7 |
| Corn Starch | 3.2 |
| Sugar | 21.6 |
| Baking powder | 1.1 |
| Xanthan | 0.05 |
| Carboxy methyl cellulose | 0.05 |
| Salt | 0.16 |
| Emulsifiers | 0.53 |
| Milk powder | 1.2 |
| Egg | 23 |
| Water | 5.4 |
| Oil Component, selected from: | 18 |
| a) Canola oil; | |
| b) Canola oil + adjuvants; or | |
| c) Conventional multipurpose shortening (mp = 38° C.) | |

Method

A concentrate was prepared by heating 20% of the oil to 90° C. Then the adjuvant(s) was added and dissolved in the oil. The concentrate was added to rest of the oil at ambient temperature and mixed to homogeneity. The resulting turbid oil composition was ready for use in the production of batter.

The batter was then prepared. The egg powder was reconstituted in water. The oil composition and the first nine components listed above were added to the bowl of a mixer (KitchenAid®) with a flat beater at low speed to obtain a smooth mixture. The reconstituted egg powder was added to the bowl, the mixer was operated for 2 minutes with the flat beater at low speed, scraped down, and mixed for an additional 2 minutes at medium speed to form a uniform batter.

The muffins were baked at 180° C. for 30 minutes.

Results

The following table shows batter specific gravity with the various oil components.

| | Specific | |
|---|---|---|
| Oil Component | gravity | |
| (% values represent percent of the oil component) | (g/ml) | Color |
| All purpose shortening (Control) | 0.93 | Pale brown |
| Canola oil | 1 | Yellow |
| Canola oil + 0.5% Stearyl Alcohol | 0.96 | Yellow |
| Canola oil + 0.25 Stearyl Alcohol + 0.25 | 0.97 | Yellow |
| Behenic Acid | | |
| Canola oil + 0.5% Stearyl Alcohol + 0.5 | 0.85 | Off-white |
| Behenic Acid | | |
| Canola oil + 1% Stearyl Alcohol + 1% | 0.82 | Off-white |
| Behenic Acid | | |
| Canola oil + 1.5% Stearyl Alcohol + 1.5% | 0.83 | Off-white |
| Behenic Acid | | |
| Canola oil + 2% Stearyl Alcohol + 2% | 0.80 | Pale brown |
| Stearic Acid | | |
| Canola oil + 4% Stearyl Alcohol + 4% | 0.80 | Pale brown |
| Stearic Acid | | |
| Canola oil + 0.5% Stearyl Alcohol + 0.5% | 0.91 | Pale brown |
| Stearic Acid | | |
| Canola oil + 0.5% Stearyl Alcohol + 0.75% | 0.86 | Off-white |
| Stearic Acid | | |
| Canola oil + 0.5% Stearyl Alcohol + 0.5% | 0.97 | Pale brown |
| Palm flake | | |

Acceptable values of specific gravity for this application are in the range of 0.8-0.9 and the batter should be off-white, indicating proper degree of aeration. As demonstrated in the above table, the desirable specific gravity and color was achieved in this system when a fatty alcohol and fatty acid were combined. The fatty alcohol (stearyl alcohol) alone gave a marginal effect compared to intact oil, as did the fatty alcohol in combination with palm flake, which consists of fully saturated triglycerides.

The following table summarizes the volume of the cake, with various mixtures of oil and adjuvant.

| | | | | % Volume |
|---|---|---|---|---|
| No. | Stearyl alcohol (%) | Behenic acid (%) | Volume | Increase |

| | | | | |
|---|---|---|---|---|
| 1 | 0 | 0 | 115 | 0 |
| 2 | 1 | 1 | 121 | 5.2 |
| 3 | 2 | 2 | 125 | 8.7 |
| 4 | 4 | 4 | 132 | 14.8 |

A volume increase was observed in all adjuvant-containing samples (Samples 2-4).

All products made with adjuvant had a finer pore structure than the control.

### EXAMPLE 6

### Yellow Cake

Composition

| | Control system | Adjuvant system |
|---|---|---|
| | % Ingredients | % Ingredients |
| White flour | 21.5 | 21.5 |
| Corn starch | 1.1 | 1.1 |
| Sugar | 23.7 | 23.7 |
| Baking powder | 1.6 | 1.6 |
| Salt | 0.8 | 0.8 |
| Emulsifiers | 1.0 | 1.0 |
| Milk powder | 2.1 | 2.1 |
| All purpose shortening | 9.7 | — |
| Oil composition (3% behenic acid + | — | 9.7 |
| 3% stearyl alcohol in soybean oil) | | |
| Egg | 19.4 | 19.4 |
| Water | 19.3 | 19.3 |

Method

All cake constituents were mixed together and the cake was baked at 180° C. for 30 minutes.

Results

A volume increase was observed in the adjuvant-containing sample: the height of the adjuvant-containing cake was 6.6 cm, compared to 5.3.cm for the control (18.8% increase). The cake made with adjuvant had a finer pore structure than the control. For dietary value, use of the adjuvant-oil composition is advantageous, since the final product has a high content of unsaturated fatty acids, whereas products made with all-purpose shortening contain high amounts of saturated and trans fatty acids, which are known to have untoward cardiovascular effects.

### EXAMPLE 7

### Yellow Cake with Fat Replacer

Composition

| | Control system | Adjuvant system |
|---|---|---|
| | % Ingredients | % Ingredients |
| White flour | 21.5 | 21.5 |
| Corn Starch | 1.1 | 1.1 |
| Sugar | 23.7 | 23.7 |
| Baking powder | 1.6 | 1.6 |
| Salt | 0.8 | 0.8 |
| Emulsifiers | 1.0 | 1.0 |
| Milk powder | 2.1 | 2.1 |
| Econa oil (diacylglycerol; DAG oil) | 9.7 | — |
| Oil Composition (3% behenic acid + | — | 9.7 |
| 3% stearyl alcohol in Econa oil) | | |
| Egg | 19.4 | 19.4 |
| Water | 19.3 | 19.3 |

Method

All cake constituents were mixed together and the cake was baked at 180° C. for 30 minutes.

Results

The batter produced with Econa oil alone was very fluid and not acceptable for baking. Presently, there is no way known to solidify such oil without changing its chemical structure and losing the structure-associated health benefits. The height of the adjuvant-containing cake was 6.0 cm, compared to 3.3 cm for the control (18.8% increase). The cake made with adjuvant had fine pores, whereas the control had unacceptable texture.

For dietary value, use of the adjuvant-oil composition in conjunction with a fat replacer having known cholesterol-lowering effects and low caloric value is highly advantageous, since the final product has a high content of unsaturated fatty acids. In contrast, products made with conventional shortening contain high amounts of saturated and trans fatty acids, which are known to have untoward cardiovascular effects. Baked products made with an adjuvant-oil composition and a fat replacer can be used in the maintenance and treatment of subjects with high cholesterol levels, obesity, diabetes and other metabolic disorders.

### EXAMPLE 8

### Yellow Cake Batter with Egg Powder

This example characterizes the concentration range of adjuvants in order to obtain a favorable batter for a particular baked good. The example demonstrates the effect of adjuvants on batter properties, and shows the superiority of the adjuvants over other options. This example also evaluates the interaction of the adjuvants with a customary baking emulsifier, distilled monoglyceride.

Composition

| | % |
|---|---|
| Sugar | 26 |
| Egg powder | 5.5 |
| Skim milk powder | 1.5 |
| Tap water | 23 |
| White flour | 30 |
| Baking powder | 0.75 |
| Salt | 0.35 |
| Oil Component, selected from: | 13 |
| a. Canola oil | |
| b. Canola oil + distilled monoglyceride | |
| c. Canola oil + adjuvants | |
| d. Canola oil + adjuvants + distilled monoglyceride | |
| e. “Control” - Conventional multipurpose shortening | |
| (mp = 38° C.) | |
| Total | 100 |

Method

The sugar, egg powder, and skim milk powder were added to the KitchenAid® mixer bowl. Water was added and the mixer was operated with the flat beater at slow speed to obtain a smooth liquid. For samples using an oil-adjuvant composition, the adjuvants were melted in 20% of the liquid oil, the rest of the oil was added, and the composition was mixed. Alternatively, all of the oil was heated with the adjuvant until melted, and then cooled.

The oil component was poured into the mixer bowl. The flour was added with the baking powder and salt. The ingredients were mixed at low speed for 2 minutes, scraped down, and mixed at medium speed for 3 minutes. The specific gravity of the batter was then measured.

Results

The following table shows batter specific gravity for the various oil components.

| | Specific | |
|---|---|---|
| Oil Component | gravity | |
| (% values represent % of the oil component) | (g/ml) | Color |
| Canola oil | 1.2 | Yellow |
| Canola oil + 1% Stearyl Alcohol + 1% Behenic | 1 | Yellow-off |
| Acid | | white |
| Canola oil + 1.5% Stearyl Alcohol + 1.5% | 0.87 | Off-white |
| Behenic Acid | | |
| Canola oil + 1.6% Stearyl Alcohol + 1.6% | 0.85 | Off-white |
| Behenic Acid | | |
| Canola oil + 2% Stearyl Alcohol + 2% Behenic | 0.83 | Off-white |
| Acid | | |
| Canola oil + 8% Distilled Monoglyceride | 0.97 | Pale brown |
| Canola oil + 4% Distilled Monoglyceride | 1.1 | Yellow |
| Canola oil + 1.6% Stearyl Alcohol + 1.6% | 0.66 | White |
| Behenic Acid + 2% Distilled Monoglyceride | | |
| Canola oil + 1.6% Stearyl Alcohol + 1.6% | 0.55 | White |
| Behenic Acid + 4% Distilled Monoglyceride | | |
| Canola oil + 1.6% Stearyl Alcohol + 1.6% | 0.54 | White |
| Behenic Acid + 8% Distilled Monoglyceride | | |
| All purpose shortening | 0.96 | Pale brown |

Acceptable values of specific gravity for yellow cake are in the range of 0.8-0.9 g/ml, and the batter should be off-white, indicating the proper degree of aeration. Lower values of specific gravity, in the range of 0.5-0.7 g/ml are typical for sponge cakes, which have a higher degree of aeration. As demonstrated in the above table, the desirable specific gravity and color for yellow cake was achieved using an oil composition including a fatty alcohol and a fatty acid at a total concentration of 2-4%. The addition of emulsifier contributed to a higher degree of aeration. In contrast, the control systems containing all-purpose shortening or emulsifier without the adjuvants had specific gravities of about 1, and the oil alone afforded specific gravity of 1.2.

### EXAMPLE 9

### Bread and Rolls

Method

This example was performed in an industrial bread factory, using the Chorleywood process with a Tweedy mixer with line output of 25 “standard” loaves of bread per minute. The regular fat used for baking “standard” bread in this facility was baking margarine, 80% fat. The standard process included manual addition of 4 kg of margarine per batch to the mixer with the yeast and other supplements. The total batch size was 270 Kg.

In the present experiments, margarine (4 kg) was replaced by 3.2 kg of an oil composition including vegetable oil plus a mixture of the adjuvants stearyl alcohol and behenic acid. In two separate experiments, corn oil and soybean oil were used. The adjuvant concentrations for production of bread were 3% stearyl alcohol+3% behenic acid, and 1.7% stearyl alcohol+1.7% behenic acid. 1.7% stearyl alcohol+1.7% behenic acid was used in the production of rolls. To prepare the 3.2 kg of oil composition, 640 g oil (about 20% of the total oil) was heated to 90° C., and the adjuvants were added with slight mixing. The adjuvants dissolved spontaneously after a few minutes. The reminder of the oil, at ambient temperature, was then added and mixed to obtain a turbid oil mixture. The oil composition was added manually into the Tweedy mixer instead of margarine.

The resulting experimental products were examined against regular products made with margarine, according to the following parameters: size/volume, texture of crumb and crust, and taste.

Results

The breads and rolls made using the oil and adjuvants were identical to standard products made with margarine in all parameters. There was no difference in the results between the adjuvant composition of 3% stearyl alcohol+3% behenic acid and 1.7% stearyl alcohol+1.7% behenic acid. Adjuvants with corn oil and soybean oil gave the same performance, indicating the generality of the beneficial effect of the adjuvant for different oils.

### EXAMPLE 10

### Sugar Cookies

Composition

| | % | grams |
|---|---|---|
| Granulated Sugar | 25.7 | 276.5 |
| Fat component selected from: | 16.0 | 172.0 |
| a) All purpose emulsified shortening; or | | |
| b) Oil plus adjuvant | | |
| Liquid whole eggs | 10.2 | 110.0 |
| Corn syrup | 8.3 | 89.0 |
| Vanilla | 0.9 | 10.0 |
| All purpose flour | 38.0 | 410.0 |
| Baking powder | 0.3 | 3.75 |
| Baking soda | 0.3 | 3.0 |
| Salt | 0.3 | 3.2 |

Method

Control cookies (with all-purpose shortening) were prepared as follows. Sugar and shortening were combined in a large bowl and beaten at medium speed for 1 minute. Eggs, corn syrup, and vanilla were added and beaten until well-blended and fluffy (2 minutes, medium speed). Flour, baking powder, baking soda, and salt were combined, gradually added to the creamed mixture at low speed (3 minutes), and mixed until well blended. The dough was refrigerated for at least one hour.

A small amount of flour was spread on a large sheet of waxed paper. One fourth of the dough was placed on the floured paper and flattened slightly by hand. The dough was turned over, covered with another large sheet of waxed paper, and rolled to ¼-inch thickness. The dough was then cut with a floured cutter and transferred to an ungreased baking sheet. The cookies were baked at 375° F. for 5 to 9 minutes, depending on their size, and cooled for 2 minutes on the baking sheet.

Cookies were prepared using an adjuvant-oil composition as follows. An adjuvant-oil composition was prepared by melting 1.5% stearyl alcohol+1.5% behenic acid+1.5% distilled monoglyceride in liquid oil. Adjuvant was melted in 20% of the liquid oil at 80° C., the rest of the oil was added, and the mixture was blended. Alternatively, all of the oil was heated to 80° C. with the adjuvant and cooled down to room temperature.

Using the baking adjuvant, the cookies were prepared in two different ways. First, the cookies were prepared in the same way as the control cookies. Second, the cookies were made by creating an emulsion first, as follows. All of the liquids (eggs, corn syrup, and adjuvant plus oil) were combined with the granulated sugar in the mixer bowl and mixed with a flat beater to get a firm emulsion (creme). Then the powders were added and mixed.

Results

The same quality of sugar cookies was obtained with the oil and adjuvant composition and with the control shortening. However, in terms of dietary value, the oil and adjuvant composition is advantageous, since the final product has a high content of unsaturated fatty acids, whereas the comparable product made with shortening contains unhealthy saturated and trans fatty acids in high amounts. Although liquid oil was used in the adjuvant system, no oiling out of the dough or the cookies was detected.

### EXAMPLE 11

### Sugar Cookies with Margarine-like Adjuvant Composition

In the production of cookies, margarine is often used instead of shortening. This example demonstrates the use of a margarine-like composition comprising liquid oil, adjuvants, water, emulsifiers, and stabilizers.

Margarine-like Adjuvant Composition

| | % |
|---|---|
| Water | 18 |
| Sisterna SP70 (emulsifier) | 1 |
| Guar gum or mix of gums such as Methocel LV15, | 0.3 |
| Acacia gum, Guar | |
| Gum & L.B.G. (stabilizer) | |
| Vegetable oil | 77.5 |
| Stearyl alcohol | 3.5 |

Sugar Cookie Composition

| | % | grams |
|---|---|---|
| Granulated Sugar | 25.7 | 276.5 |
| Margarine-like adjuvant composition | 16.0 | 172.0 |
| Liquid whole eggs | 10.2 | 110.0 |
| Corn syrup | 8.3 | 89.0 |
| Vanilla | 0.9 | 10.0 |
| All purpose flour | 38.0 | 410.0 |
| Baking powder | 0.3 | 3.75 |
| Baking soda | 0.3 | 3.0 |
| Salt | 0.3 | 3.2 |

Method

The margarine-like composition was prepared as follows. Stearyl alcohol was added to the oil and the mixture was heated to 75-80° C. with stirring. The emulsifier SP70 and stabilizer were added to the water, and the aqueous mixture was stirred at room temperature for 15 minutes to yield a homogeneous paste. The aqueous mixture was then heated gradually to 65-80° C., while stirring was continued. The warm oil mixture was then added gradually to the water phase, with stirring, and the final mixture was further mixed for 30 seconds at 1500 RPM, and then poured into containers and refrigerated.

Sugar cookies were prepared using the margarine-like composition as follows. The sugar and margarine-like adjuvant composition were combined in a large bowl and beaten at medium speed for 1 minute. The eggs, corn syrup, and vanilla were added to the mixture and beaten until well-blended and fluffy (2 minutes, medium speed). The flour, baking powder, baking soda, and salt were combined, gradually added to the creamed mixture at low speed (3 minutes), and mixed until well blended.

A small amount of flour was spread on a large sheet of waxed paper. One fourth of the dough was placed on the paper and flattened slightly. The dough was turned over, covered with another large sheet of waxed paper, and rolled to ¼-inch thickness. The dough was then cut with a floured cutter and transferred to an ungreased baking sheet.

The cookies were baked at 375° F. for 5 to 9 minutes, depending on their size, and cooled for 2 minutes on the baking sheet.

Results

The quality of the cookie, as measured by its physical properties, taste and mouth feel, was identical to comparable cookies made with shortening or butter. For dietary value, use of the margarine-like adjuvant-oil composition is advantageous, since the final product has a high content of unsaturated fatty acids, whereas comparable products made with shortening contain high amounts of saturated and trans fatty acids, which have known untoward cardiovascular effects. This difference is of particular importance in cookies, which typically contain high amounts of fat.

### EXAMPLE 12

### Cake without Flour

Composition

| | % |
|---|---|
| Eggs | 35 |
| Sugar | 22.5 |
| Xanthan gum (optional) | 0.1 |
| Corn starch | 5 |
| Fat component, selected from | 10 |
| a) Butter; or | |
| b) Oil and adjuvant composition (2.5% stearyl alcohol + | |
| 2.5% behenic acid in oil) | |
| Nut meal | 7.5 |
| Bitter chocolate | 20 |

Method

The control cake (with butter) was prepared as follows. An egg yolk compound was formed by mixing the egg yolk and sugar, and then adding the corn starch, xanthan gum, and nut meal. A chocolate compound was formed by melting the chocolate in a hot water bath, then adding the fat component and mixing to uniformity. The chocolate compound was added to the egg yolk compound and mixed to uniformity. The egg white was whipped to form a fluffy whip, which was added to the mixture with slow mixing to yield a batter. The batter was poured into a cake form and baked for 45 minutes at 180° C.

The cake with oil and adjuvant composition was prepared as follows. The oil and adjuvant composition was prepared by melting 2.5% stearyl alcohol+2.5% behenic acid in liquid oil at 80° C. Two methods were found efficient for producing the batter. The first method was similar to the preparation of the control cake. The second method was an easy procedure without egg separation, performed as follows. Whole eggs were poured into the mixer bowl. Sugar, starch powder, and xanthan gum were then added to the eggs, and the mixture was whipped for 2 minutes. The oil and adjuvant composition was added, and the mixture was whipped with a wire beater for 5 minutes at high speed. The nut meal and the melted chocolate were added, and the mixture was further whipped for 2 minutes at medium speed, to uniformity. The batter was poured into a cake form and baked for 45 minutes at 180° C.

Results

The quality of the cake made with oil and adjuvant, as measured by its physical properties, taste, and mouth feel, was identical to the comparable cake made with butter. For dietary value, use of the adjuvant-oil composition is advantageous, since the final product has a high content of unsaturated fatty acids, whereas the same product made with butter contains high amounts of saturated fatty acids, which have known untoward cardiovascular effects.

### EXAMPLE 13

### Pie Crust

Composition

| | Control | Adjuvant |
|---|---|---|
| Ingredients | Composition | Composition |

Fat component selected from:

| | | |
|---|---|---|
| a) Emulsified shortening | 30.9% | — |
| b) 1.5% stearyl alcohol + 1.5% | — | 23% |
| behenic acid + | | |
| 1.5% distilled monoglyceride in soybean oil | | |
| Pastry flour | 51.4% | 57% |
| Salt | 1.0% | 1.1% |
| Whey | 1.5% | 1.6% |
| Water (ice cold) | 15% | 16.6% |

Method

The fat component was mixed for 1½ minutes on an Artofex mixer. The pastry flour, salt, and whey were added to the mixer and mixed for 1½ minutes. The water was added and mixed for one minute.

The pie crust was baked at 400° F. for 14 minutes or until golden brown.

Results

The control and adjuvant pie crusts had similar structure and cookie-like taste. However, less adjuvant-oil composition than shortening was required to obtain a similar product in the dough and the pie crust. In terms of dietary value, the adjuvant-oil composition is advantageous, since the final product has a high content of unsaturated fatty acids, whereas the comparable product made with shortening contains high amounts of unhealthy saturated and trans fatty acids.

### EXAMPLE 13

### Icing

Composition

| | | grams |
|---|---|---|
| A | Sisterna sucrose ester SP 70 (emulsifier) | 12 |
| | Guar Gum (stabilizer) | 4 |
| | Granulated sugar | 522 |
| | Water | 227 |
| B | Adjuvant - oil composition (3% stearyl alcohol and 3% | 398 |
| | behenic acid in oil) | |
| C | Powdered sugar | 840 |
| | NFDM (non-fat dry milk) | 85 |
| | Salt | 7 |

Method

The water-soluble ingredients (A) were mixed together to dissolve the granulated sugar and yield a homogenous phase at 60° C. The warm adjuvant-oil composition (B) was poured into the water phase with rapid agitation, to create an oil-in-water emulsion, which was then cooled to room temperature. The emulsion was transferred to the mixer bowl and whipped with the dry ingredients (C).

The amount of stabilizer and the proportion between the dissolved sugar and powdered sugar was adjusted in order to modify the viscosity of the icing.

Results

The specific gravity of the creme made with the adjuvant-oil composition was 0.64 g/ml, compared with 0.74 g/ml for the same product with control shortening. The color was lighter for the adjuvant-oil creme than the color of a shortening-based creme.

As will be apparent to one of skill in the art from a reading of this disclosure, the invention can be embodied in forms other than those specifically disclosed above without departing from the spirit or essential characteristics of the invention. The particular embodiments of the invention described above are, therefore, to be considered as illustrative and not restrictive. The scope of the invention is as set forth in the appended claims, rather than being limited to the examples contained in the foregoing description.

What is claimed is:

1. An oil composition for use in baking comprising a mixture of between about 70% and about 99.95% of an oil and between about 0.05% and about 30% of a baking adjuvant selected from the group consisting of fatty alcohols having a molecular weight of at least 200 Da, fatty acids having a molecular weight of at least 200 Da, and combinations thereof.

2. The oil composition of claim 1, wherein the oil is selected from the group consisting of liquid oils, semi-solid and solid fats, fat replacers, and combinations thereof in any proportion.

3. The oil composition of claim 2, wherein the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil.

4. The oil composition of claim 2, wherein the oil comprises an oil or fat selected from the group consisting of a partially or fully hydrogenated oil, an animal-derived fat, margarine, coconut oil, palm oil, palm kernel oil, butter fat, and cocoa fat.

5. The oil composition of claim 2, wherein the oil comprises a fat replacer selected from the group consisting of diglycerides, triglycerides, combinations of short- and long-chain fatty acids, sucrose esters, and edible indigestible hydrophobic materials.

6. The oil composition of claim 2, wherein the oil comprises at least about 25% of a fat replacer having a caloric value of less than 6.9 kcal/g.

7. A baked good comprising the oil composition of claim 6.

8. The baked good of claim 7 selected from the group consisting of cake, muffin, tart, bread, bun, bagel, cookie, biscuit, wafer, pie, pizza, pita, doughnut, pancake, cracker, and pastry.

9. The oil composition of claim 1, wherein the adjuvant comprises a fatty alcohol having a molecular weight of at least about 200 Da.

10. The oil composition of claim 9, wherein the fatty alcohol has between about 15 and about 50 carbon atoms in its hydrocarbon chain.

11. The oil composition of claim 9, wherein the fatty alcohol is selected from the group consisting of cetyl alcohol, stearyl alcohol, arachidyl alcohol, behenyl alcohol (docosanol), octacosanol, and1-triacontanol.

12. The oil composition of claim 9, wherein the fatty alcohol has a branched hydrocarbon chain having at least one alkyl group side chain.

13. The oil composition of claim 12, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

14. The oil composition of claim 9, wherein the fatty alcohol has at least one additional hydroxyl group linked to its hydrocarbon chain.

15. The oil composition of claim 9, wherein the fatty alcohol has at least one double bond in its hydrocarbon chain.

16. The oil composition of claim 1, wherein the adjuvant comprises a fatty acid having a molecular weight of at least about 200 Da.

17. The oil composition of claim 16, wherein the fatty acid has between about 15 and about 50 carbon atoms in its hydrocarbon chain.

18. The oil composition of claim 16, wherein the fatty acid is selected from the group consisting of hexadecanoic (palmitic) acid (C16), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30).

19. The oil composition of claim 16, wherein the fatty acid has a branched hydrocarbon chain having at least one alkyl group side chain.

20. The oil composition of claim 19, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

21. The oil composition of claim 16, wherein the fatty acid has at least one additional hydroxyl group linked to its hydrocarbon chain.

22. The oil composition of claim 16, wherein the fatty acid has at least one double bond in its hydrocarbon chain.

23. The oil composition of claim 1, wherein the adjuvant comprises at least one fatty alcohol and at least one fatty acid in a ratio between about 2:8 and about 8:2.

24. A baked good comprising the oil composition of claim 23.

25. The baked good of claim 24 selected from the group consisting of cake, muffin, tart, bread, bun, bagel, cookie, biscuit, wafer, pie, pizza, pita, doughnut, pancake, cracker, and pastry.

26. The oil composition of claim 23, wherein the fatty alcohol is stearyl alcohol and the fatty acid is behenic acid.

27. A baked good comprising the oil composition of claim 26.

28. The baked good of claim 27 selected from the group consisting of cake, muffin, tart, bread, bun, bagel, cookie, biscuit, wafer, pie, pizza, pita, doughnut, pancake, cracker, and pastry.

29. The oil composition of claim 1, comprising between about 80% and about 99.95% of the oil, and between about 0.05% and about 20% of the adjuvant.

30. The oil composition of claim 1, comprising between about 0.05% and about 10% of the adjuvant.

31. The oil composition of claim 1, further comprising at least one additive selected from the group consisting of emulsifiers, stabilizers, anti-oxidants, flavors, and colorants.

32. A batter or dough comprising the oil composition of claim 1.

33. A baked good comprising the oil composition of claim 1.

34. The baked good of claim 33 selected from the group consisting of cake, muffin, tart, bread, bun, bagel, cookie, biscuit, wafer, pie, pizza, pita, doughnut, pancake, cracker, and pastry.

35. The baked good of claim 33 having a reduced caloric value.

36. The baked good of claim 33 having a cholesterol-lowering effect.

37. An icing comprising the oil composition of claim 1.

38. A baking kit comprising the oil composition of claim 1 and additional baking ingredients.

39. The baking kit of claim 38, wherein the additional baking ingredients comprise ingredients for making at least one baked good selected from the group consisting of cake, muffin, tart, bread, bun, bagel, cookie, biscuit, wafer, pie, pizza, pita, doughnut, pancake, cracker, and pastry.

40. An adjuvant-oil concentrate for use in baking comprising a mixture of between about 5% and about 50% of an oil and between about 50% and about 95% of a baking adjuvant selected from the group consisting of fatty alcohols having a molecular weight of at least 200 Da, fatty acids having a molecular weight of at least 200 Da, and combinations thereof.

41. The concentrate of claim 40, wherein the adjuvant comprises at least one fatty alcohol and at least one fatty acid in a ratio between about 2:8 and about 8:2.

42. The concentrate of claim 41, wherein the fatty alcohol is stearyl alcohol and the fatty acid is behenic acid.

43. A baking kit comprising the concentrate of claim 40 and additional baking ingredients.

44. The baking kit of claim 43, wherein the additional baking ingredients comprise ingredients for making at least one baked good selected from the group consisting of cake, muffin, tart, bread, bun, bagel, cookie, biscuit, wafer, pie, pizza, pita, doughnut, pancake, cracker, and pastry.

45. A method of reducing or preventing an increase in a blood cholesterol level comprising administering to a subject in need thereof an effective amount of the oil composition of claim 1.

46. A method of treating or preventing hyperlipidemia or hypercholesterolemia comprising administering to a subject in need thereof an effective amount of the oil composition of claim 1.

47. A method of promoting weight loss or preventing weight gain comprising administering to a subject in need thereof an effective amount of the oil composition of claim 1.

48. An emulsion comprising between about 40% and about 90% of the oil composition of claim 1 and between about 10% and about 60% water.

49. The emulsion of claim 48, further comprising at least one additive selected from the group consisting of emulsifiers, stabilizers, flavors, and colorants.

50. A batter or dough comprising an oil composition, said oil composition comprising a mixture of an oil and an adjuvant selected from the group consisting of fatty alcohols having a molecular weight of at least 200 Da, fatty acids having a molecular weight of at least 200 Da, and combinations thereof, wherein the adjuvant forms between about 0.01% and about 5% of the dough or batter and the oil forms between about 0.5% and about 50% of the dough or batter.

51. The batter or dough of claim 50, wherein the oil is selected from the group consisting of liquid oils, semi-solid and solid fats, fat replacers, and combinations thereof in any proportion.

52. The batter or dough of claim 51, wherein the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil.

53. The batter or dough of claim 51, wherein the oil comprises an oil or fat selected from the group consisting of a partially or fully hydrogenated oil, an animal-derived fat, margarine, coconut oil, palm oil, palm kernel oil, butter fat, and cocoa fat.

54. The batter or dough of claim 51, wherein the oil comprises a fat replacer selected from the group consisting of diglycerides, triglycerides, combinations of short- and long-chain fatty acids, sucrose esters, and edible indigestible hydrophobic materials.

55. The batter or dough of claim 51, wherein the oil comprises at least about 25% of a fat replacer having a caloric value of less than 6.9 kcal/g.

56. The batter or dough of claim 50, wherein the adjuvant comprises at least one fatty alcohol and at least one fatty acid in a ratio between about 2:8 and about 8:2.

57. The batter or dough of claim 56, wherein the fatty alcohol is stearyl alcohol and the fatty acid is behenic acid.

58. The batter or dough of claim 50, wherein the adjuvant comprises a fatty alcohol having between about 15 and about 50 carbon atoms in its hydrocarbon chain.

59. The batter or dough of claim 58, wherein the fatty alcohol is selected from the group consisting of cetyl alcohol, stearyl alcohol, arachidyl alcohol, behenyl alcohol (docosanol), octacosanol, and 1-triacontanol.

60. The batter or dough of claim 58, wherein the fatty alcohol has a branched hydrocarbon chain containing at least one alkyl group side chain.

61. The batter or dough of claim 60, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

62. The batter or dough of claim 58, wherein the fatty alcohol has at least one additional hydroxyl group linked to its hydrocarbon chain.

63. The batter or dough of claim 58, wherein the fatty alcohol has at least one double bond in its hydrocarbon chain.

64. The batter or dough of claim 50, wherein the adjuvant comprises a fatty acid having between about 15 and about 50 carbon atoms in its hydrocarbon chain.

65. The batter or dough of claim 64, wherein the fatty acid is selected from the group consisting of hexadecanoic (palmitic) acid (C1 6), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30).

66. The batter or dough of claim 64, wherein the fatty acid has a branched hydrocarbon chain containing at least one alkyl group side chain.

67. The batter or dough of claim 66, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

68. The batter or dough of claim 64, wherein the fatty acid has at least one additional hydroxyl group linked to its hydrocarbon chain.

69. The batter or dough of claim 64, wherein the fatty acid has at least one double bond in its hydrocarbon chain.

70. The batter or dough of claim 50, further comprising at least one additive selected from the group consisting of emulsifiers, stabilizers, anti-oxidants, flavors, and colorants.

71. A baked good comprising the batter or dough of claim 50.

72. The baked good of claim 71 selected from the group consisting of cake, muffin, tart, bread, bun, bagel, cookie, biscuit, wafer, pie, pizza, pita, doughnut, pancake, cracker, and pastry.

73. The baked good of claim 71 having a reduced caloric value.

74. The baked good of claim 71 having a cholesterol-lowering effect.

75. A baked good comprising an adjuvant selected from the group consisting of fatty alcohols having a molecular weight of at least 200 Da, fatty acids having a molecular weight of at least 200 Da, and combinations thereof.

76. The baked good of claim 75, wherein the adjuvant forms between about 0.05% and about 5% of the baked good.

77. The baked good of claim 75, wherein the adjuvant comprises at least one fatty alcohol and at least one fatty acid in a ratio between about 2:8 and about 8:2.

78. The baked good of claim 77, wherein the fatty alcohol is stearyl alcohol and the fatty acid is behenic acid.

79. The baked good of claim 75 selected from the group consisting of cake, muffin, tart, bread, bun, bagel, cookie, biscuit, wafer, pie, pizza, pita, doughnut, pancake, cracker, and pastry.

80. A baked good comprising an oil composition, said oil composition comprising a mixture of an oil and an adjuvant selected from the group consisting of fatty alcohols having a molecular weight of at least 200 Da, fatty acids having a molecular weight of at least 200 Da, and combinations thereof, wherein the adjuvant forms between about 0.01% and about 5% of the baked good and the oil forms between about 0.5% and about 50% of the baked good.

81. The baked good of claim 80, wherein the oil is selected from the group consisting of liquid oils, semi-solid and solid fats, fat replacers, and combinations thereof in any proportion.

82. The baked good of claim 81, wherein the oil comprises a liquid oil selected from the group consisting of a vegetable oil, an unsaturated oil, a monounsaturated fatty acid-rich oil, an oleic acid-rich oil, an omega-3 fatty acid rich oil, and a marine oil.

83. The baked good of claim 81, wherein the oil comprises an oil or fat selected from the group consisting of a partially or fully hydrogenated oil, an animal-derived fat, margarine, coconut oil, palm oil, palm kernel oil, butter fat, and cocoa fat.

84. The baked good of claim 81, wherein the oil comprises a fat replacer selected from the group consisting of diglycerides, triglycerides, combinations of short- and long-chain fatty acids, sucrose esters, and edible indigestible hydrophobic materials.

85. The baked good of claim 81, wherein the oil comprises at least about 25% of a fat replacer having a caloric value of less than 6.9 kcal/g.

86. The baked good of claim 80, wherein the adjuvant comprises at least one fatty alcohol and at least one fatty acid in a ratio between about 2:8 and about 8:2.

87. The baked good of claim 86, wherein the fatty alcohol is stearyl alcohol and the fatty acid is behenic acid.

88. The baked good of claim 80, wherein the adjuvant comprises a fatty alcohol having between about 15 and about 50 carbon atoms in its hydrocarbon chain.

89. The baked good of claim 88, wherein the fatty alcohol is selected from the group consisting of cetyl alcohol, stearyl alcohol, arachidyl alcohol, behenyl alcohol (docosanol), octacosanol, and 1-triacontanol.

90. The baked good of claim 88, wherein the fatty alcohol has a branched hydrocarbon chain containing at least one alkyl group side chain.

91. The baked good of claim 90, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

92. The baked good of claim 88, wherein the fatty alcohol has at least one additional hydroxyl group linked to its hydrocarbon chain.

93. The baked good of claim 88, wherein the fatty alcohol has at least one double bond in its hydrocarbon chain.

94. The baked good of claim 80, wherein the adjuvant comprises a fatty acid having between about 15 and about 50 carbon atoms in its hydrocarbon chain.

95. The baked good of claim 94, wherein the fatty acid is selected from the group consisting of hexadecanoic (palmitic) acid (C16), heptadecanoic (margaric) acid (C17), octadecanoic (stearic) acid (C18), eicosanoic (arachidic) acid (C20), docosanoic (behenic) acid (C22), tetracosanoic (lignoceric) acid (C24), octacosanoic acid (C28), and triacontanoic acid (C30).

96. The baked good of claim 94, wherein the fatty acid has a branched hydrocarbon chain containing at least one alkyl group side chain.

97. The baked good of claim 96, wherein the alkyl group side chain is selected from the group consisting of methyl, ethyl, n-propyl, i-propyl, n-butyl, t-butyl, i-butyl, n-pentyl, i-pentyl, t-pentyl, neo-pentyl, and linear or branched hexyl, heptyl, octyl, nonyl, decyl, lauryl, cetyl, and stearyl groups.

98. The baked good of claim 94, wherein the fatty acid has at least one additional hydroxyl group linked to its hydrocarbon chain.

99. The baked good of claim 94, wherein the fatty acid has at least one double bond in its hydrocarbon chain.

100. The baked good of claim 80, further comprising at least one additive selected from the group consisting of emulsifiers, stabilizers, anti-oxidants, flavors, and colorants.

101. The baked good of claim 80 selected from the group consisting of cake, muffin, tart, bread, bun, bagel, cookie, biscuit, wafer, pie, pizza, pita, doughnut, pancake, cracker, and pastry.

102. The baked good of claim 80 having a reduced caloric value.

103. The baked good of claim 80 having a cholesterol-lowering effect.

104. A method of reducing or preventing an increase in a blood cholesterol level comprising administering to a subject in need thereof an effective amount of the baked good of claim 80.

105. A method of treating or preventing hyperlipidemia or hypercholesterolemia comprising administering to a subject in need thereof an effective amount of the baked good of claim 80.

106. A method of promoting weight loss or preventing weight gain comprising administering to a subject in need thereof an effective amount of the baked good of claim 80.

107. A method of making a batter or dough comprising:
a) combining an oil with an adjuvant selected from the group consisting of fatty alcohols having a molecular weight of at least 200 Da, fatty acids having a molecular weight of at least 200 Da, and combinations thereof, thereby forming an oil composition; and
b) combining the oil composition with additional baking ingredients, thereby forming a batter or dough.

108. The method of claim 107, wherein combining the adjuvant with the oil comprises:
a) heating the oil above the melting temperature of the adjuvant;
b) adding the adjuvant to the heated oil; and
c) mixing the adjuvant with the oil.

109. The method of claim 107, wherein combining the adjuvant with the oil comprises:
a) mixing the adjuvant with a portion of the oil, thereby forming an adjuvant-oil concentrate; and
b) mixing the adjuvant-oil concentrate with the remainder of the oil, thereby forming the oil composition.

110. The method of claim 107, further comprising combining a fatty alcohol having a molecular weight of at least 200 Da and a fatty acid having a molecular weight of at least 200 Da in a ratio of between about 2:8 and about 8:2, thereby forming the adjuvant.

111. The method of claim 107, wherein the oil composition comprises between about 70% and about 99.95% of the oil and between about 0.05% and about 30% of the adjuvant.

112. A method of making a baked good comprising:
a) combining an oil with an adjuvant selected from the group consisting of fatty alcohols having a molecular weight of at least 200 Da, fatty acids having a molecular weight of at least 200 Da, and combinations thereof, thereby forming an oil composition;
b) combining the oil composition with additional baking ingredients, thereby forming a batter or dough; and
c) baking the batter or dough, thereby forming a baked good.

113. The method of claim 112, wherein combining the adjuvant with the oil comprises:
a) heating the oil above the melting temperature of the adjuvant;
b) adding the adjuvant to the heated oil; and
c) mixing the adjuvant with the oil.

114. The method of claim 112, wherein combining the adjuvant with the oil comprises:
a) mixing the adjuvant with a portion of the oil, thereby forming an adjuvant-oil concentrate; and
b) mixing the adjuvant-oil concentrate with the remainder of the oil, thereby forming the oil composition.

115. The method of claim 112, further comprising combining a fatty alcohol having a molecular weight of at least 200 Da and a fatty acid having a molecular weight of at least 200 Da in a ratio of between about 2:8 and about 8:2, thereby forming the adjuvant.

116. The method of claim 112, wherein the oil composition comprises between about 70% and about 99.95% of the oil and between about 0.05% and about 30% of the adjuvant.

117. The method of claim 112, wherein the baked good has reduced caloric value.
